(19) United States
(12) Patent Application Publication
(10) Pub. No.: US 2011/0135556 A1
(43) Pub. Date: Jun. 9, 2011

(54) METHOD AND DEVICE FOR PRODUCING ENERGY, DME (DIMETHYL ETHER) AND BIO-SILICA USING CO2-NEUTRAL BIOGENIC REACTIVE AND INERT INGREDIENTS

(75) Inventors: Oliver Neumann, Offenbach (DE); Peter Mehrling, Darmstadt (DE)

(73) Assignee: SPIRIT OF TECHNOLOGY AG, Offenbach am Main (DE)

(21) Appl. No.: 13/057,282

(22) PCT Filed: Aug. 3, 2009
(86) PCT No.: PCT/EP09/60018

§ 371 Date: Feb. 3, 2011

(30) Foreign Application Priority Data
Aug. 7, 2008 (DE) 10 2008 036 734.6

(51) Int. Cl. C01B 33/20; C07C 41/01; C10J 3/46; B01J 8/18; C01B 33/12

(52) U.S. Cl. 423/326; 568/671; 48/197 R; 422/141; 422/119; 423/335

(57) ABSTRACT

Processes and facilities for the production of biological synthesis gases and/or a synthetic propellant, in particular DME (dimethyl ether), and/or biological silica using biogenic input materials and comprising the following steps:
Allothermal gasification of the biogenic input material by means of impulse burners for the integrated generation of process heat in a fluidized bed gasifier
Gasification of inert pyrolytic coke from the first gasification step in a second gasification step preferably operated in parallel according to the principle of the expanded or circulating fluidized bed using oxygen/steam as gasification agents
Combination of at least part of the gasification products from the two gasifiers for common processing

### FIELD OF THE INVENTION

Based on gasification processes published by SPOT as patent applications 10 2007 004 294.0/10 2006 017 355.4/10 2006 039 622.7/10 2006 019 999.5/10 2006 017 353.8 the present invention describes the SPOT combined gasification process for extending the range of biogenic input materials to those which, due to their natural consistency, form an inert pyrolytic coke in the first, simultaneously performed step of the gasification reaction (allothermal and autothermal), the processing of the biological synthesis gas generated during the SPOT gasification process or the SPOT combined gasification process to form dimethyl ether (DME) using the modular process routes already described in the application via the isolated intermediate methanol or the non-isolated intermediate product, as well as the preferred use of this product in the INCOX100 process for the highly efficient generation of electric power.

The gasification process integrates the relevant gasification processes theoretically and practically available to create a new process that makes it possible to gasify all conceivable biogenic input materials in a highly efficient manner (rate of mass turnover clearly above 90%) while considering highest demands to economic efficiency.

The development of thermal gasification processes essentially brought forth three different types of gasifiers: the entrained gasifier, the fixed bed gasifier and the fluidized bed gasifier. In addition, dependant on the source of the enthalpy flow required for the gasification reaction, the gasification processes are divided into autothermal processes, in which the reaction enthalpy is generated in the same process by reacting the input materials to form CO₂ and H₂O (combustion), and allothermal gasification processes, in which the enthalpy flow necessary for the gasification reaction is not generated during the process but spatially separated and fed to the gasification process by means of convection, heat transfer (SPOT process) or radiation.

Information on fluidized bed gasification can be obtained from the below references which are part of the present application: “High-Temperature Winkler Gasification of Municipal Solid Waste”; Wolfgang Adlhoch, Rheinbraun AG, Hisaaki Sumitomo Heavy Industries, Ltd.; Joachim Wolff, Karsten Radtke (speaker), Krupp Uhde GmbH; Gasification Technology Conference; San Francisco, Calif., USA; Oct. 8-11, 2000; Conference Proceedings. Information on circulating fluidized bed gasification in combined systems can be obtained from the below references which are part of the present application: “Dezentrale Strom-und Warmeerzeugung auf Basis Biomasse-Vergasung”, R. Rauch, H. Hofbauer; lecture at the university of Leipzig 2004; “Zirkulierende Wirbelschicht, Vergasung mit Luft, Operation Experience with CfB-Technology for Waste Utilisation at a Cement Production Plant”, R. Wirthwein, P. Scur, K.-F. Scharf, Rüdersdorfer Zement GmbH; H. Hirschfelder—Lurgi Energie und Entsorgungs GmbH, 7^{th} International Conference on Circulating Fluidized Bed Technologies; Niagra Falls, May 2002.

The SPOT developments restrict the reaction pressure of the gasification process to the low-pressure range since due to the special reaction kinetics of the gasification process the space-time yield of the major process facilities are virtually independent of pressure; therefore, the benefit of using high pressure does not appropriately correspond to the technical effort of a pressurized gasification. Due to technical and economical reasons the multi-step processes reported in the references comprising basically the entrained gasifier with an upstream pyrolysis step known from the gasification of coal dust and heavy fuel oil (Carbo V, Forschungszentrum Karlsruhe) seem to be completely unsuitable for commercial processes for the gasification of biogenic input materials.

The fluidized bed gasifiers can be divided into two processes: the circulating fluidized bed gasifier and the stationary fluidized bed gasifier.

In early 2002, an allothermal circulating fluidized bed gasification facility was put into service in Güssing (Austria). There, biomass is gasified in a fluidized bed using steam as oxidant. Some of the charcoal produced in the fluidized bed is combusted in a second fluidized bed to provide heat for the gasification process.

During this gasification using steam a product gas is generated. The disadvantages are high acquisition costs for industrial manufacturing equipment and excessive expenses for process control.

To overcome the problems of the prior art the applicant has already consigned various applications regarding this field the disclosure of which is part of the present application. These applications are 10 2006 017 353.8; 10 2006 017 355.4; 10 2006 019 999.5; 10 2006 022 265.2; 10 2006 039 622.7.

It is known from these applications that biomass is gasified in a fluidized bed using steam as reaction and fluidizing medium. However, here the fluidized bed is a stationary fluidized bed with two specifically developed impulse burners making an indirect heat supply to the fluidized bed inside the reactor possible. Hereinafter, this process is referred to as SPOT process.

Autothermal gasification is characterized by the lack of distinct temperature and reaction zones. Since the fluidized bed consists of an inert bed material, the simultaneous proceeding of the individual partial reactions and a homogeneous temperature (approx. 800° C.) are guaranteed. The process is technically feasible and characterized by high economic efficiency. The acquisition costs are below those of the above-mentioned types of gasifiers.

### The SPOT Combined Gasification Process

The range was extended to biogenic input materials tending to form an inert coke in the pyrolysis step of the gasification. The process is characterized in that the material discharged from the fluidized bed—a mixture of bed material, ash and pyrolytic coke—is conveyed into a second, autothermally operated stationary or expanded/circulating fluidized bed, either directly or following screening and examination to separate carbon and fine particles. The product gas, a synthesis gas rich in CO, is added to the major gas stream prior to gas cooling, the coarse particles of the ash are returned to the allothermal gasifier of the SPOT gasification system and the fine particles, a high-quality biological silica raw material, are discharged.

As in the main process, the combined product gas generated during allothermal and autothermal gasification of pyrolytic coke fractions is subjected to dry dusting, cooling and compression to be conveyed to the other processes as a compressed biological synthesis gas.

### Processes

It can be stated as a result that via the process routes “SPOT gasification by means of SPOT gasification processes” and/or “SPOT combined gasification process” a synthesis gas produced from biogenic input materials is made available, from which, in a selective synthesis using various process steps, the synthetic propellant DME (dimethyl ether) is obtained in a high yield. In this process, up to 41 tons of synthetic propellant (DME) may be produced from 100 tons of input material.

The process of the present invention is based on the above-mentioned patent applications, an allothermal fluidized bed gasification process comprising a special impulse burner for the generation of the reaction heat required for the gasification reaction that is intended for the use of internal gas or so-called off gases originating from the processing methods processing the biological synthesis gas to form the finished products.

This gasifier is extended to a parallel gasification step in which the pyrolytic coke produced during the gasification reaction is reacted to form a synthesis gas using steam and oxygen as gasification agents. As a SPOT combined gasification process, the entire gasification process is designed such that the portion of this autothermal gasification step is minimized, which is already demanded by economic efficiency. This autothermal partial process is incorporated by discharging the ash/bed material of the allothermal step and considering the synthesis gas generated with regard to the allothermally generated synthesis gas; therefore, further conditioning (cooling etc.) of the synthesis gas and treatment of the bed material are performed at the same time. This autothermal gasifier is an integral part of the SPOT combined gasification process.

Using this array, the reaction rates of biogenic input materials forming (intermediate) inert pyrolytic coke as gasification intermediate can be increased to values clearly above 95%. Due to their silicate content the incurring ashes thus remain an excellent high-quality biological silica raw material.

This gasification process comprises in-situ desulfurization (patent application 10 2007 004 294.9), hot gas purification (patent application 10 2006 017 353.8), removal of halogens by means of adsorption (patent application 10 2007 004 294.0), single- or multi-step fine cleaning using multi-cyclones and sintered metal filters and quenching, in which traces of condensable aliphatic and aromatic hydrocarbons are washed out by means of a non-aqueous washing liquid. The deposited substances are returned to the gasifier and reacted to form a synthesis gas which is then cooled for the subsequent compression steps.

For the use of biogenic gasification substances tending to form an inert coke in the pyrolysis step of the gasification, the mass stream cyclically discharged from the fluidized bed of the SPOT allothermal gasifier, a mixture of bed material, ash of the input materials and pyrolytic coke, is conveyed in a second gasification step—either directly or following screening and examination to separate carbon and fine particles—into an authothermal gasifier operating according to the principle of the circulating fluidized bed. This gasifier is operated at near-atmospheric pressure and temperatures of up to 1000° C. and more using oxygen/steam as gasification agents. Here, the pyrolytic coke is reacted. The product gas, a synthesis gas rich in CO, is added to the major gas stream prior to cooling and the coarse particles of the ash are conveyed into the allothermal gasifier of the SPOT gasification system. The fine particles, a high-quality biological silica raw material, are discharged.

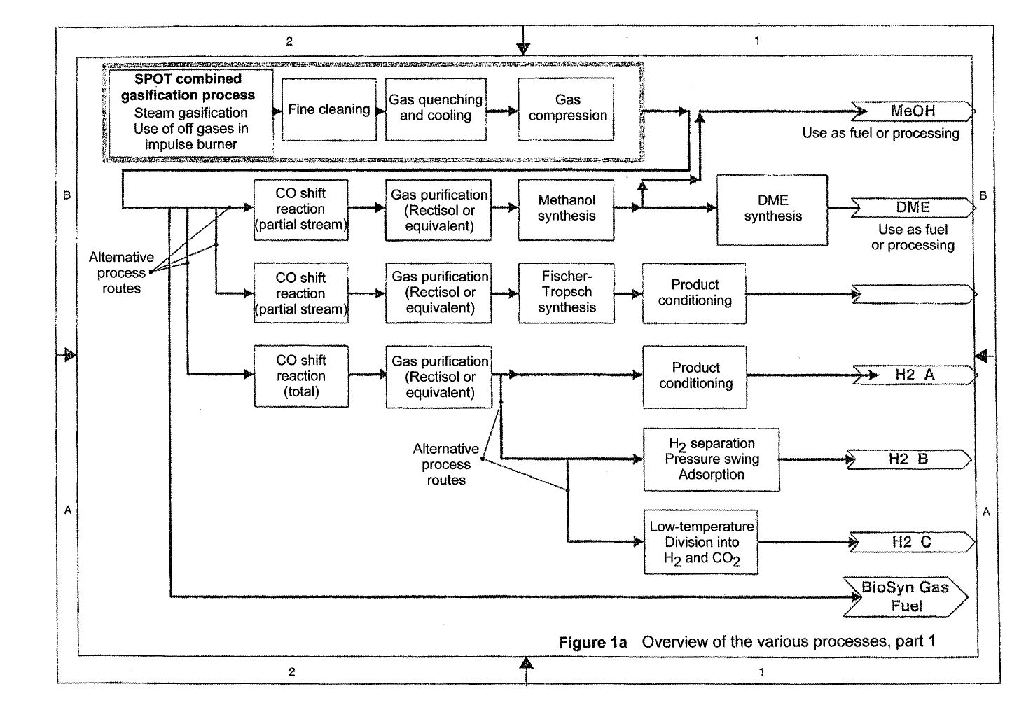

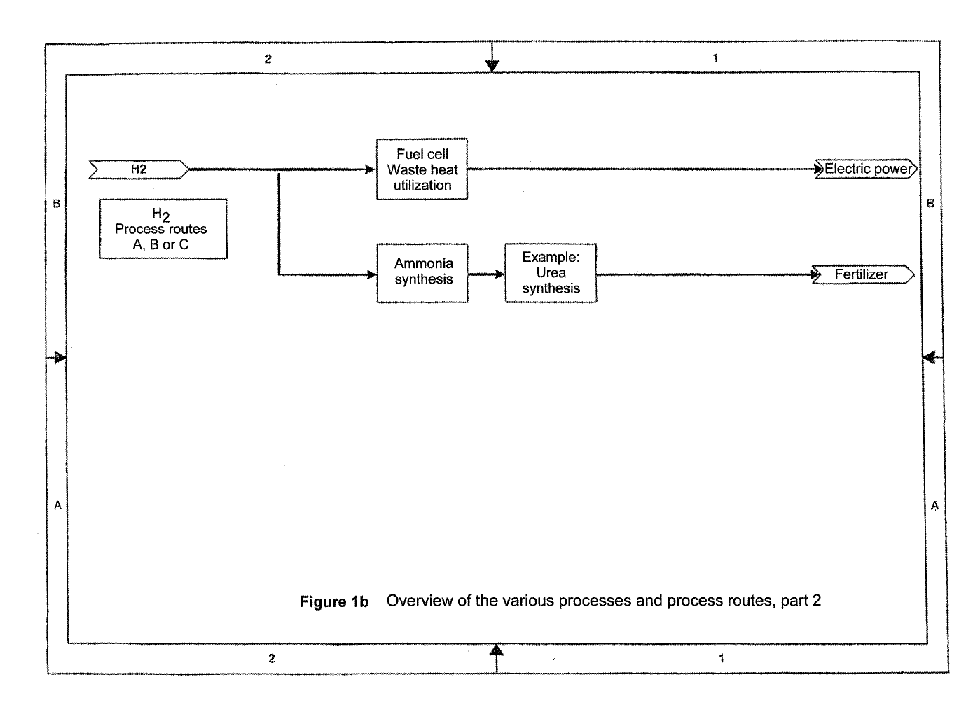

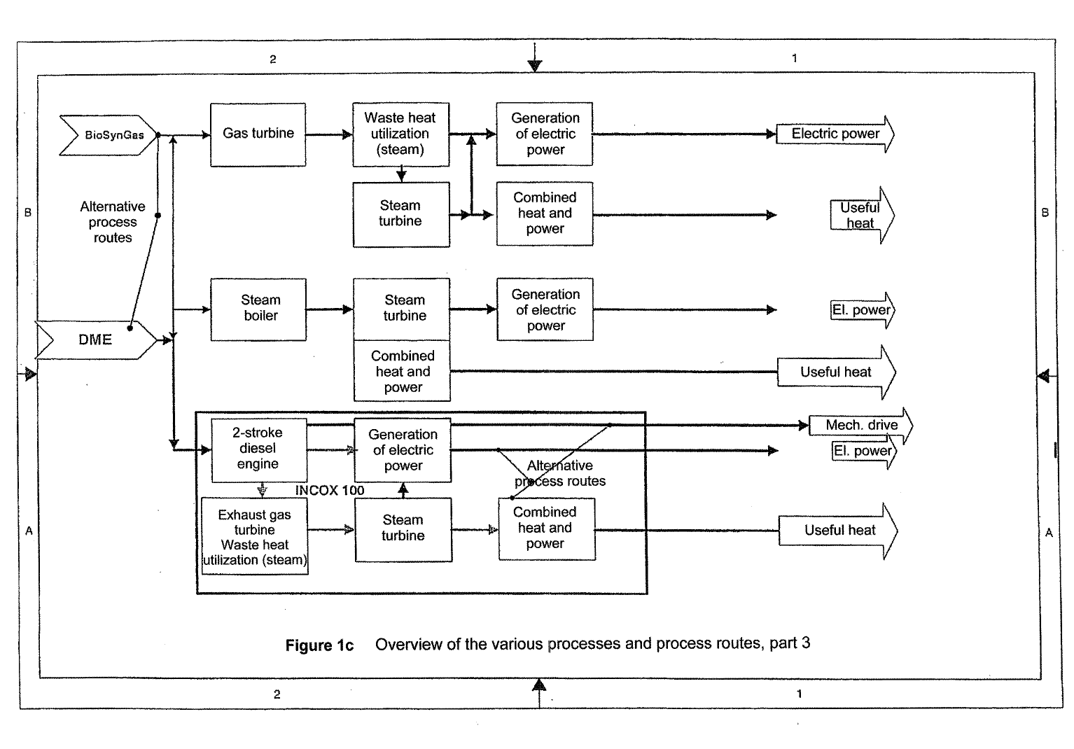

The present invention integrates the process routes according to FIG. 1 for the generation of chemical substances, synthetic propellants and hydrogen, the generation of electric or mechanical power by the combustion of the synthesis gas in gas turbines, steam generators and engines or the use of, for example, hydrogen in fuel cells as described in the patent application 10 2007 004 294.0 as well as the use of synthetic fuel, in particular DME, for the generation of electric power in the INCOX100 process.

### OVERVIEW OF THE INVENTION

The object of the present invention is the production of the synthetic propellant DME, the generation of electric/mechanical power in the INCOX100 process based on biogenic input materials and the production of biological synthesis gas generated in the SPOT gasification process and the SPOT combined gasification process also described in the present invention. In the present application emphasis—regarding propellants—is put on DME showing a very high yield and economic efficiency due to its intermediate methanol. A yield of 41 tons per 100 tons of input material is possible. The advantage of this process route as compared to competing processes is its simplicity and the uniform, high-yield product. Apart from the use as a propellant it can be employed as liquid gas substitute and chemical raw material.

DME is highly suitable to be used for INCOX100 (internal combustion box), which is currently available as a 2-stroke engine with a mechanical power of 100 MW/h. When using this technology for the generation of electricity, an aggregated power of up to 1000 MW/h is easily possible for power plants and obvious for the driving of ships. As a rule, DME is also suitable for 4-stroke combustion engines. For the sake of completeness, the use in gas turbines and steam generators is set forth.

The SPOT combined gasification process allows the use of an extraordinarily broad range of biogenic input materials and extends the usability of the underlying SPOT gasification process according to the present invention to inert biogenic input materials tending to intermediately form inert pyrolytic coke, which can only inadequately be reacted to form a biological synthesis gas in the allothermal gasification step of the SPOT process due to the restricted maximum gasification temperature in this process.

The object of the present invention is the gasification of the entire range of possible biogenic input materials, including those forming an inert pyrolytic coke when generating the biological synthesis gas and its reaction products. This object is resolved by a process and a facility exhibiting the characteristics of the independent claims.

The underlying SPOT gasification process of the allothermal steam gasification by means of impulse burners is suitable for various types of renewable raw materials, including the patented SPOT power greenies, to convert the described biological synthesis gas, which is suitable as an input material for the generation of energy and the combustion in steam generators, gas turbines or thermal engines with internal combustion, by chemical synthesis to produce propellants and chemical products. The usability of the process is considerably extended to the SPOT combined gasification process developed for the first time.

The SPOT process allows the large-scale generation of energy, propellants and chemical intermediates from renewable raw materials or biomass, which in turn are starting materials for the whole range of products currently manufactured on the crude oil basis. The proposed process routes shown in the below description are thus examples of both the possibilities and the key processes forming the interface between the renewable resources and further chemical processes based on a closed circuit.

Input materials are all renewable raw materials which—and this is the only theoretical restriction—can be brought to a residual moisture content of preferably less than 35 mass percent by using an amount of energy that is clearly smaller than the chemical energy bound by the substance or the corresponding caloric value. Thus, due to the basic reaction conditions, the process is not suitable for biomasses that are highly aqueous or contain only few mass percents of solids (e.g. liquid manure).

The embodiment of the gasification comprising two gasification steps allows the use of input materials forming highly inert pyrolytic coke.

By-products and regenerative biomasses, power greenies, animal feedstuff, agricultural refuse, waste from the food industry and wood of all kinds and types can be reacted by this extended process (the specific adjustments such as conditioning of the input materials, conveyance into the gasification reactor and bed management are marginal) to form an amply usable intermediate. In addition, the performance of the gasification process as an allothermal gasification process in conjunction with the SPOT combined gasification process allows the production of a synthesis gas which otherwise is only available by gasification using oxygen in a highly efficient manner. The latter approach comprises the technically complex and—due to the thermodynamic transformation processes—energetically ineffective generation of electric power and the subsequent production of oxygen.

The gasification of the partial stream, i.e. of the pyrolytic coke insufficiently reacted in the allothermal gasifier, does not essentially change this statement.

This concept thus allows the generation of all energies required for the production in a CO₂-neutral manner, i.e. as net CO₂ consumers, for example by urea synthesis, which increases the CO₂ portion of the synthesis gas which is then reacted as well.

The inventions described below relate to the SPOT combined gasification process and the process route circuit allowing the use of the biological synthesis gas of the SPOT gasifier to generate energy, propellants and chemical products.

These routes are characterized by the integrated use of the purge gas (basically methane) as a fuel for generating the reaction heat of the SPOT gasification process, energetic efficiency and high material utilization of the input materials. The use of the above-mentioned synthetic propellant DME for the generation of electric power in the INCOX100 process is part of the invention.

The following details are considered:
1. SPOT combined gasification process
2. Use of off/purge gas and other combustible gases produced in the downstream processes for generating the process heat of the gasification reaction in the impulse burners of the allothermal SPOT gasification process
3. Mechanical, physical gas purification including gas compression
4. Generation of DME based on biological synthesis gas
5. Generation of mechanical power (driving power) and electric power using DME as input material
6. Typical performance characteristics of the INCOX100 process
7. Generation of electricity by using DME in the INCOX100 process

### Production of the Following Products Using Biological Synthesis Gas as Starting Material (FIG. 1)

Methanol
DME via methanol as isolated or non-isolated intermediate
Gasoline/diesel via methanol as isolated or non-isolated intermediate
H₂ as input material for fuel cells or as reactant for various chemical syntheses, e.g. ammonia synthesis and urea synthesis as a reaction product (fertilizer production), olefin syntheses, hydrogenating syntheses etc.
Electricity (i.e. mechanical or electric power) by direct combustion of the biological synthesis gas and use in gas turbines or combustion in the internal combustion box (thermal engine with internal combustion), also for generating mechanical power and primarily electric power
Biological silica is an environmentally friendly raw material with a high silicon content that is obtained by the gasification of biogenic agricultural by-products. Due to its high-quality chemical, mineralogical and physical properties the silica (SiO₂) extracted from ash is required as a necessary auxiliary for the production of steel, ceramics, mortar or cement, fertilizer, paper, plastics, cosmetics etc.

Below the modular process routes already set forth in the patent applications are explained again.

In the pure gas CO shift process the previously compressed biological synthesis gas is adjusted such regarding the molar CO/H₂ portion that the optimum ratio for further synthesis is obtained.

As a rule—with the exception of H₂ generation—this CO shift is a partial stream shift. The process integration allows to minimize the partial stream to be converted to obtain the required gas composition.

The synthesis gas adjusted to the requirements of the subsequent synthesis by means of its CO/H₂ ratio is now subjected to a gas purification step as shown in the gas purification step of patent application 10 2007 004 294.0 to eliminate the CO₂ content and various trace substances acting as catalyst toxins (e.g. sulfur components). This embodiment is an example of a number of possible process circuits reducing CO₂ to a portion tolerable for the subsequent syntheses and removing all trace substances acting as catalyst toxins.

As an alternative to these applications of the synthesis gas, the use of the biological synthesis gas-based synthetic propellant DME and subsequently methanol, diesel or gasoline according to the 2-stroke or 4-stroke principle is reasonable due to the extraordinarily high efficiency of INCOX100.

As a result of the synthesis of methanol and DME an off gas is produced that may be used either directly or following the separation of hydrogen from this gas mixture, e.g. by the pressure swing process, to generate the reaction heat required for the gasification in the integrated impulse burners.

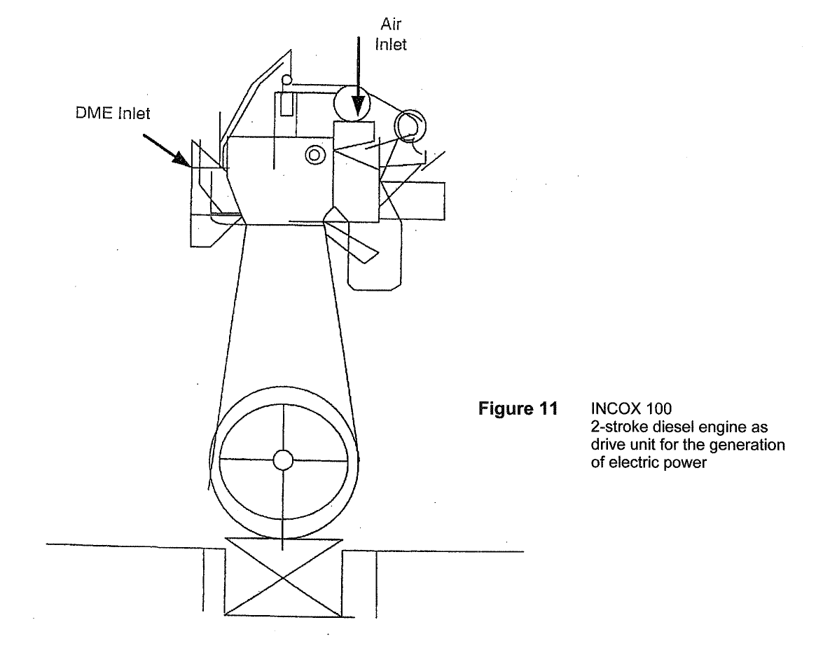

In FIG. 11 the use as a ship drive is shown (2-stroke, slow-speed large engine with a torque range of approx. 100 rpm). Regarding the use as an electric power plant with an available power of up to 100 MW/h, power plant blocks with a power of up to 1000 MW/h are easily possible. Due to their exhaust gas utilization (exhaust gas turbines and utilization of exhaust gas heat by means of steam turbines) these engines have an efficiency of clearly more than 70%. For this high rate of mechanical power to be available for the generator, this combination has proven to be the technically superior concept in the field of combined heat and power regarding the effective fraction of the low-temperature heat.

### DESCRIPTION OF THE FIGURES

### Figures

FIG. 1a-1c depict an overview of the different process routes showing the production of DME and the use of this propellant in INCOX100 for both the generation of electricity and as a fuel for gas turbines, steam generators and engines in general

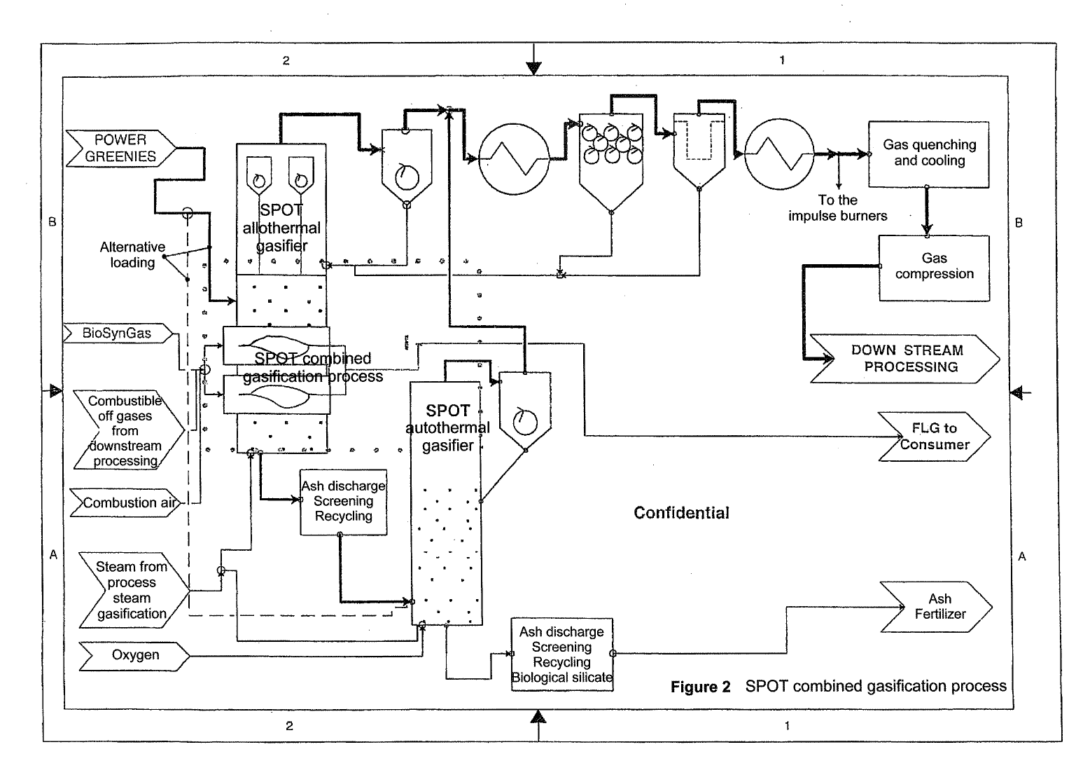

FIG. 2: 2-step SPOT combined gasification process

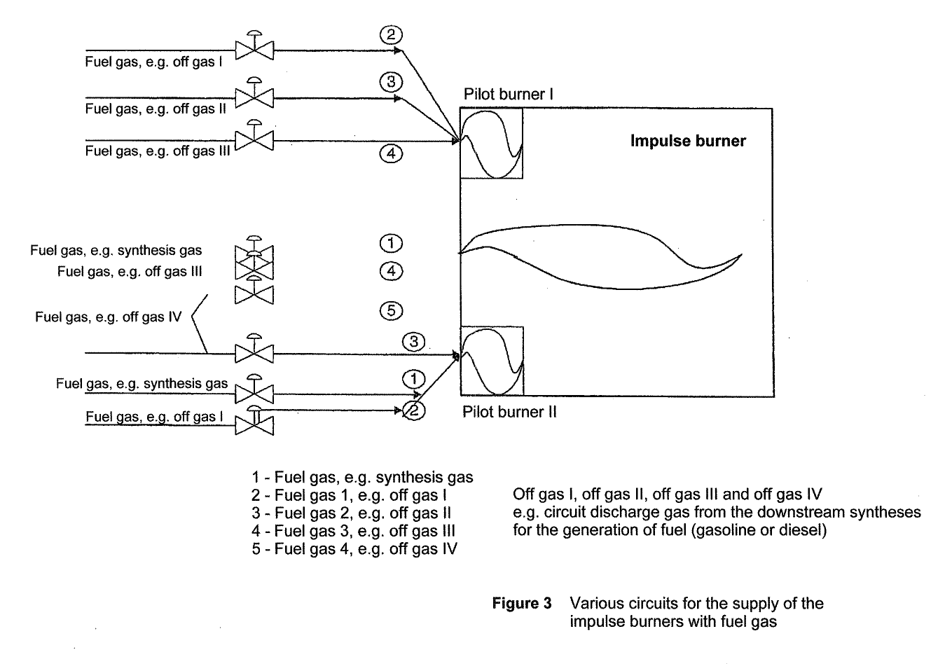

FIG. 3 shows various circuits for the supply of impulse burners with propellant

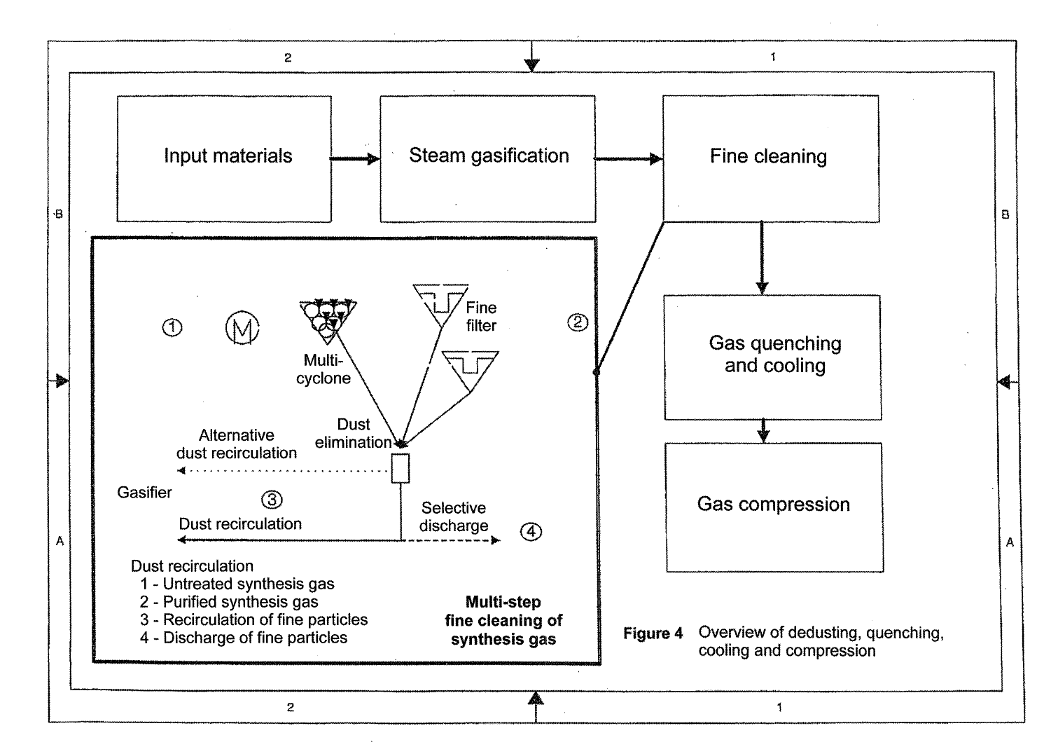

FIG. 4 shows an overview of the dusting, quenching, cooling and compression

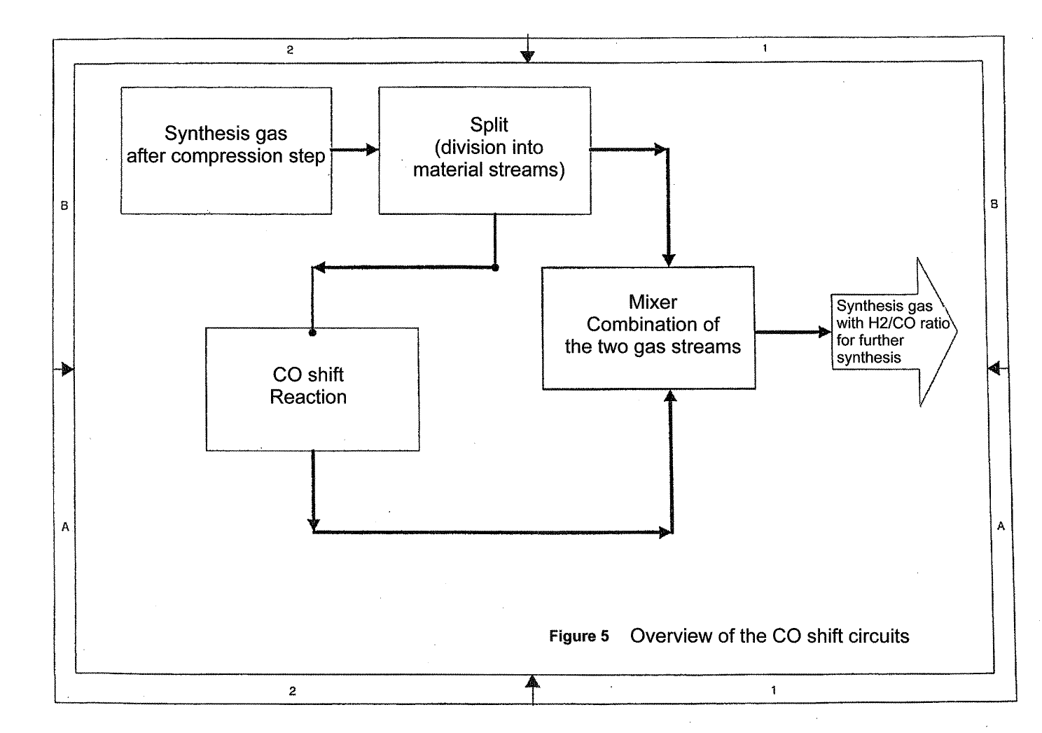

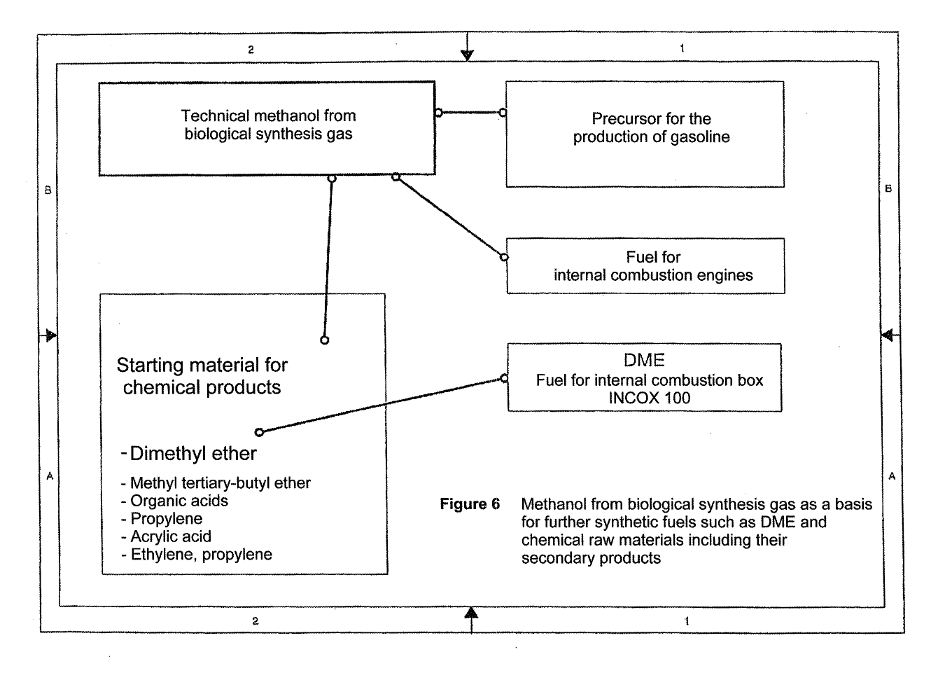

FIG. 6 shows an overview of the use (or input) of methanol produced from the biological synthesis gas as an intermediate for the production of synthetic aliphatic hydrocarbons as well as the production of DME (dimethyl ether) as universal propellant and precursor for the synthesis of various chemical products

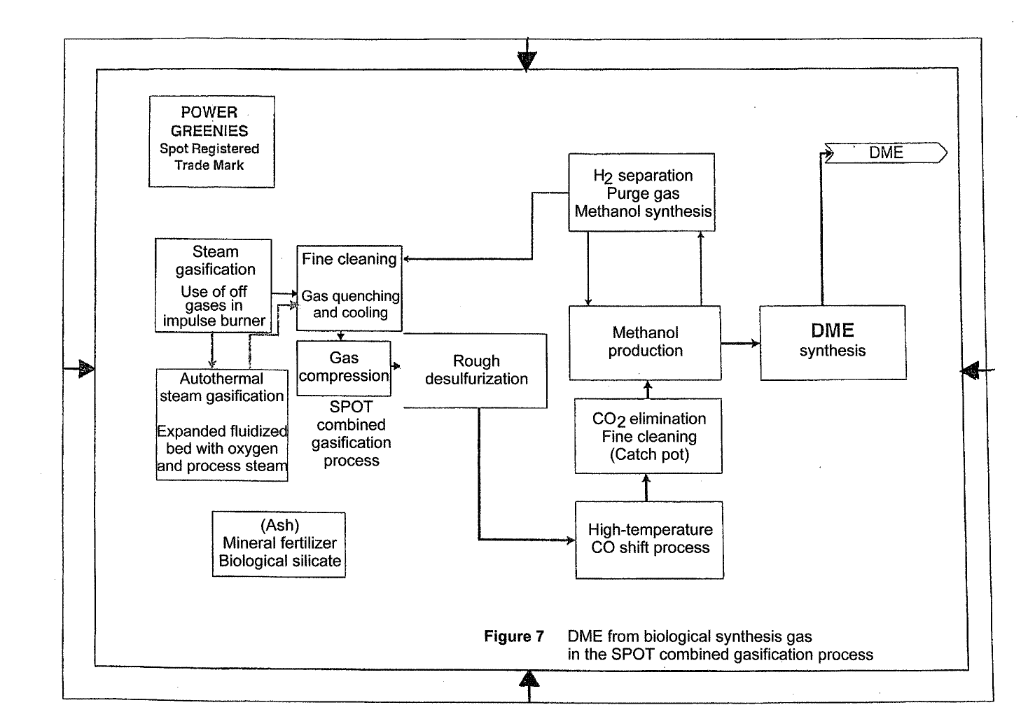

FIG. 7: DME synthesis from synthesis gas of the SPOT gasification processes

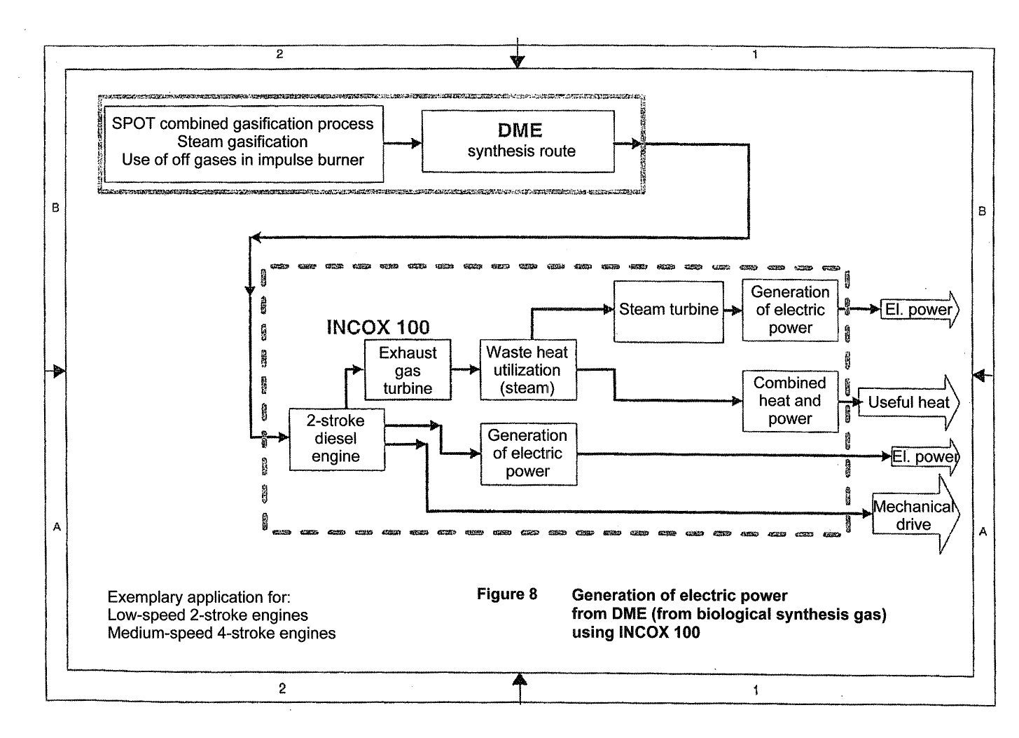

FIG. 8: INCOX100 electric power in general

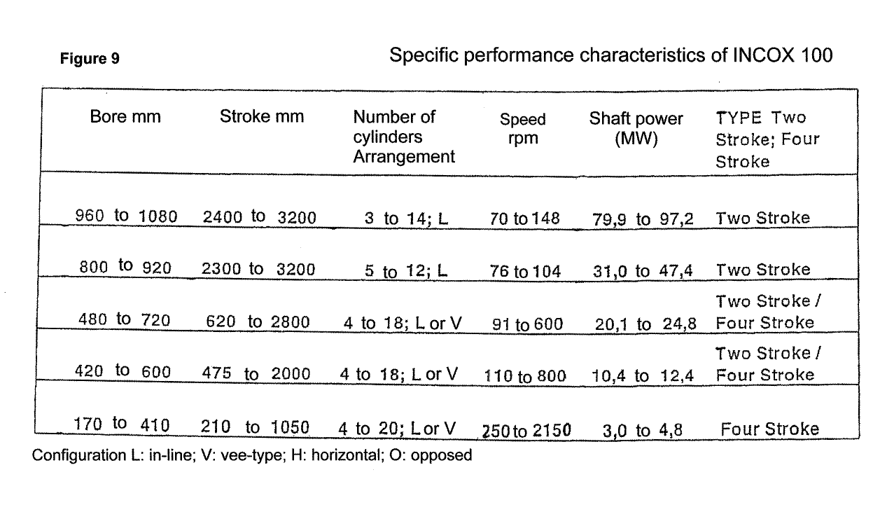

FIG. 9: INCOX100 specific performance characteristics

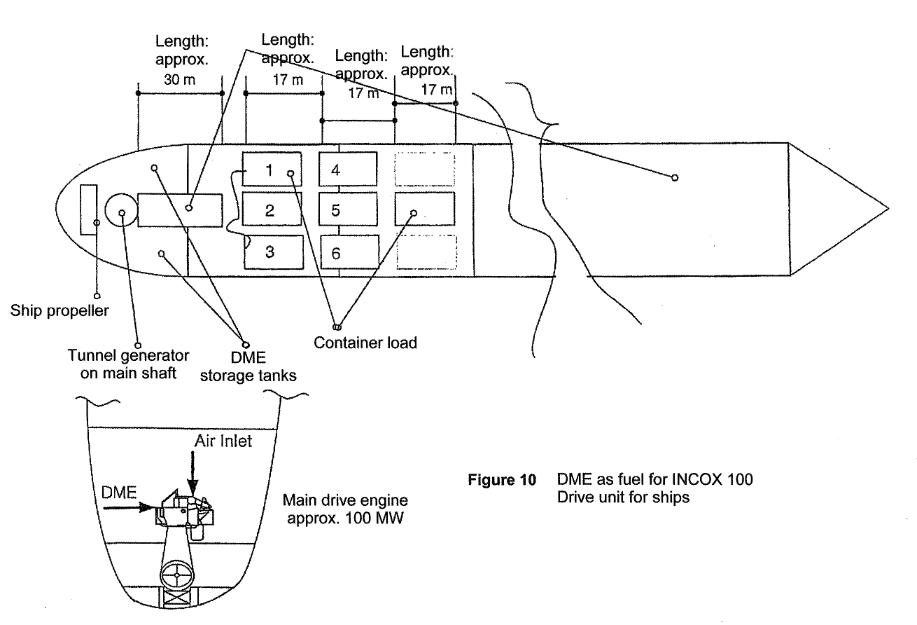

FIG. 10: INCOX100 use as ship drive

FIG. 11: INCOX100 section as an example of the stationary generation of electric power

### DETAILED DESCRIPTION OF THE EMBODIMENTS

Hereinafter, the SPOT combined gasification process, the process route for the production of the synthetic propellant DME and the use thereof in both the INCOX100 process and gas turbines, steam generators etc. for the generation of electric and/or mechanical (shaft) power as can be seen in FIG. 1a-1c are described.

The object of the present invention is an extension of this technology to extend the range of input materials to biogenic input materials tending to form an inert coke in the pyrolysis step of the gasification. The process is characterized in that the material discharged from the fluidized bed—a mixture of bed material, ash and pyrolytic coke—is conveyed into a second, autothermal, stationary or expanded/circulating fluidized bed gasifier (either directly or following screening and examination to separate carbon and fine particles), in which the pyrolytic coke is reacted to form the synthesis gas using oxygen and steam as gasification agents. This product gas, a synthesis gas rich in CO, is added to the major gas stream from the allothermal gasification prior to gas cooling, the coarse particles of the ash are returned to the allothermal gasifier of the SPOT gasification system and the fine particles—a natural fertilizer—are discharged. In FIG. 2 the circuit of the 2-step SPOT gasification process is shown.

The below description is an example of the array of a second gasification step connected in parallel to a SPOT allothermal gasifier. The choice of the flow rate and flow rate ratio between allothermal gasification and autothermal gasification is not restricted and depends on the specific conditions of use, i.e. the biogenic input materials used. The present invention is not restricted regarding the flow rate ratio of the two gasification types.

In the gasification system, the input material “power greenies” is preferably conveyed in the first step into the allothermal SPOT gasifier with integrated generation of process heat by means of impulse burners (for details see the above-mentioned SPOT patent applications). Following rough dusting, the product gas generated—the biological synthesis gas—is subjected to cooling and a first fine cleaning to reach the downstream processes following gas quenching and compression.

In the case of input materials forming inert pyrolytic coke the ash introduced with the input material is discharged together with the unreacted pyrolytic coke and screened and/or examined during conditioning for the high-carbon (or entire) fraction to be conveyed to the second gasification step of the gasification process.

In this step performed according to the principle of the expanded or circulating fluidized bed the pyrolytic coke is reacted at temperatures of up to 1500° C. using oxygen/steam as gasification agents to form a synthesis gas rich in CO (due to the low H₂ content of the input material). The process includes the addition of the original input materials to this coke (the autothermal gasification of the coke at higher temperatures is the actual goal of this process step) as far as this is required by the gasification processes (mass and heat balance, minimum flow rate). Since this second gasifier is operated in an inert bed, the gasification temperature of the selected material should be clearly above that of the first step, i.e. the material should not agglomerate, bake or stick under these conditions. The gasification agents are distributed via the established distribution system of the SPOT allothermal step and the bed material is returned from the expanded fluidized bed by means of a (highly charged) cyclone with a dynamic sealing at the solid side to restrict the quantity.

For example, the product gas is added to the biological synthesis gas produced during allothermal gasification and then used as a unit. Although the separate use is also part of the present invention, it is of secondary interest regarding practical use.

The utilization of residual gases (off/purge gases) of the subsequent processes (downstream processes) as fuels for the impulse burners is described below.

The embodiment of the SPOT combined gasification process allows the use of the high-caloric off gases produced in the process routes described below (as often generated in the circuits of these processes in the form of residual gases or purge gases) as a fuel for the impulse burner system (impulse burners and integrated pilot burners). The result of this operation is the increase in total efficiency of the process steps and the optimum utilization of the renewable raw material used. The high-caloric off gas is used to generate the required reaction heat for the gasification reactions. To this end, the impulse burners and the integrated pilot burners are equipped with several independent supply strands for the various fuels and exhaust gases. By incorporating the off gases the processes are rendered an integrated member of the SPOT combined gasification process and the process steps become a direct and unmistakably linked unit (see FIG. 3, various circuits for the supply of impulse burners with fuel). Another possible variant is the use of these off gases as fuels for the impulse burners after having been conditioned, e.g. by separation of H₂, which may be used for example as a reactant in the synthesis of methanol.

The technical equipment allows the standard start-up of the gasification facility using biological synthesis gas, crude oil and propane as well as the various exhaust gases of the subsequent processes. In the case of gasifiers connected in parallel the concept also allows the start-up using biological synthesis gas from the parallel gasifiers. For an extension of the range of applications of the input materials required for the start-up of the gasifiers (fuels of the impulse burners) the use of DME (dimethyl ether) generated from the biological synthesis gas is possible and in accordance with the present invention.

It is intended to subject the gas to a mechanical purification and fine cleaning using multi-cyclones and sintered metal filters (gas conditioning prior to compression of the biological synthesis gas).

It is required to compress the biological synthesis gas and, due to thermodynamic and mechanical engineering requirements, cool the product gas to a temperature range of preferably less than 100° C. In this temperature range the condensable hydrocarbons contained in traces in the biological synthesis gas condense, in particular during the start-up.

The concept described below—the mechanical cleaning of the biological synthesis gas in the described process step (FIG. 4) and the cooling in the process step using preferably oil, biological diesel or other suitable washing and cooling media—guarantees the required purity and cooling. The concept avoids the accumulation of residual material streams that are technically unusable. The details of this invention are explained in the patent applications 10 2007 004 294.0 and 10 2006 017 353.8.

Subsequently, the gas is compressed in the pressure steps required for the subsequent processes. The compression of the gas depends on the requirements of the subsequent processes and becomes necessary when the process pressure of these subsequent process steps is above that of the gasification. This step may be directly integrated into the process step or performed separately. The processes can be described in detail as follows:
Subsequent processes at the pressure level of the gasification unit: no-pressure and near-atmospheric pressure processes such as the combustion of the product gas in steam generators or the firing of industrial furnaces (e.g. rotary kilns for the manufacture of quicklime, cement furnaces etc.)
Subsequent processes operated at elevated pressure
Processes with integrated compressors (e.g. turbo-chargers): An example is the use of the biological synthesis gas in gas turbines.
Processes with external compression to increase the admission pressure of the synthesis gas to the reaction pressure required for the subsequent processes. To this end, synthesis facilities are used which, as a rule, are operated at a pressure level in the range of 20 to 30 bar.

Another aspect is the production of synthesis gas for the generation of energy (hydrogen), synthetic propellants such as DME and chemical products via direct synthesis or by using methanol as an intermediate, as already applied for. One of the most important reaction products of the biological synthesis gas regarding the use as a propellant is DME (dimethyl ether). This product is either available via the isolated methanol of the methanol synthesis or via the non-isolated intermediate methanol. FIG. 7 shows an overview of the process route of the DME production from biological synthesis gas. Apart from CO conversion and gas purification the process routes comprise the entire gas generation according to the SPOT process or the SPOT combined gasification process including purification steps and compression.

The following description is focused on FIG. 7 and in particular on the production of DME (shown example: reaction product of the methanol produced during methanol synthesis) as well as its use as a propellant in INCOX100, gas turbines and, for the sake of completeness, steam generators.

Since further processes such as various processes for the generation of H₂ including its use in fuel cells, the production of ammonia and ammonia-based reaction products such as the fertilizer urea and the Fischer-Tropsch process including its variants and reaction products have already been submitted as patent application (patent application 10 2007 004 294.0), no further explanations are given here.

It is essential to emphasize DME synthesis from methanol based on the synthesis of methanol that has already been mentioned in the above-mentioned patent application. This selective, high-turnover process is available in two versions, via the isolated (condensed) methanol and directly via the product gas of the methanol synthesis, gaseous methanol. Both are catalytic processes.

The following description of the DME synthesis based on biological synthesis gas is according to FIG. 7.

Following the compression step at a pressure of approx. 20 bar to eliminate traces of the sulfur compounds (H₂S and others) the biological synthesis gas is roughly desulfurized. The sulfur traces contained in the biological synthesis gas prior to this process are absorbed for example by contact with an iron chelate solution, catalytically oxidized to form sulfur and converted, for example, in a special partial stream CO shift process (high-temperature CO shift). After this conversion the molar H₂/CO ratio is set; following the elimination of the major CO₂ content in a chemical washing process and the installation of a catch pot (zinc oxide catalyst) for the protection of the catalyst the conditions for the subsequent processes (methanol synthesis and subsequent process of DME synthesis) are met.

After further compression of the converted synthesis gas free of CO₂ and trace substances it is added to the circuit gas stream and the hydrogen separated from the off (purge) gases of the methanol synthesis, for example by means of a pressure swing process, and conveyed to the methanol synthesis. The raw methanol obtained in this synthesis is subsequently reacted in another process step to form DME.

To set the balance, i.e. restrict the portion of unreacted components, the purge gas from the methanol synthesis is separated and, following separation of the hydrogen portion, conveyed to the SPOT gasification process for the generation of process heat.

The generation of electric power from biogenic input materials, allothermal gasification in the SPOT gasification process and direct combustion of the conditioned gas after it has been purified, compressed and finally cooled in the combustion chamber(s) of gas turbines, steam generators, directly fired industrial furnaces and internal combustion engines (large engines) have been described in detail in the patent application 10 2007 004 294.0.

However, the synthetic propellant DME based on biological synthesis gas is a carbon-neutral input material that can be used in steam generators and gas turbines, for example as an industrial heat source for decentralized heat generation, in the INCOX100 process for the generation of electric power and for driving ships, as a propellant in vehicles and as a substitute for liquid gas. These applications are shown in FIG. 1c.

The present invention describes the use of the product generated on the basis of biological synthesis gas in the INCOX100 process.

INCOX100 is a process the key part of which is the internal combustion box. This box is a combustion unit with internal combustion, integrated compression of combustion air and exhaust gas expansion. This facility is available as a 2- or 4-stroke version with a power of 100 MW/h. In both cases the energy of the flue gas stream from the combustion is optimally used after internal expansion by exhaust gas turbines, waste heat utilization, steam generation and utilization in steam turbines as well as optionally by combined heat and power to generate mechanical and/or electric power. Due to the technically available modular power of 100 MW/h the use of this technology for the generation of electric power makes electric power plants (INCOX100 power plants) with 1000 MW/h and more possible.

Further essential aspects of the INCOX100 process are as follows: the combustion air is charged to obtain an optimum efficiency of the engine, a technically available high internal pressure is achieved during the combustion at the beginning of the work stroke, and the energy of the flue gas is utilized after combustion in the exhaust gas turbine (expansion turbine compressing the combustion air, using the residual expansion work to drive a generator and additionally using the enthalpy of the combustion gases/flue gases to generate steam).

In addition, it is intended to uncouple heat for heating purposes (combined heat and power). Due to the described utilization of exhaust gases this process has a mechanical efficiency of clearly more than 70%. At least theoretically, this efficiency may be increased again by more than 15 efficiency points when integrating combined heat and power. This concept according to the present invention comprising combined heat and power is characterized by the high mechanical efficiency (thus indicating the very high electrical efficiency) which exceeds that of current power plants by factor 2. This is the technical top concept in the field of combined heat and power, since the effective fraction of low temperature/heat for heating purposes is lower, although it does not have to be consumed permanently.

1. A process for the production of biological synthesis gases and/or a synthetic propellant, in particular DME (dimethyl ether), and/or biological silica using biogenic input materials and comprising the following steps:
Allothermal gasification of the biogenic input material by means of impulse burners for the integrated generation of process heat in a fluidized bed gasifier
Gasification of inert pyrolytic coke from the allothermal gasification step in a second gasification step preferably operated in parallel according to the principle of the expanded or circulating fluidized bed using oxygen/steam as gasification agents; and
Combination of at least part of the gasification products from the two gasifiers for common processing.

2. The process according to claim 1, in which discharged material is screened and/or examined to separate carbon and/or fine particles to isolate biological silicate contained in ash.

3. The process according to claim 1, in which coarse particles of ash are returned to the allothermal gasifier and/or fine particles of ash are discharged as a high-quality biological silicate product.

4. The process according to claim 1, in which the process for the production of biological synthesis gases further comprises one or more of the following steps:
In-situ desulfurization;
Hot gas purification;
Removal of halogens by adsorption;
1- or 2-step fine cleaning using multi-cyclones and sintered metal filters;
Quenching using a non-aqueous washing liquid to wash out traces of condensable aliphatic and/or aromatic hydrocarbons; or
Gas cooling for subsequent compression steps.

5. The process according to claim 1, in which dimethyl ether (DME) is produced from generated biological synthesis gas via the intermediate methanol.

6. The process according to claim 1, in which the inert pyrolytic coke formed during allothermal gasification (due to the biogenic input material) is reacted in a second gasification step using a mixture of oxygen/steam as gasification agents.

7. The process according to claim 1, in which, with regard to a molar CO/H₂ portion, previously compressed biological synthesis gas in a pure gas CO shift process is set such that an optimum ratio for further synthesis is achieved.

8. A facility for the production of biological synthesis gases and/or a synthetic propellant, in particular DME (dimethyl ether), using biogenic input materials and comprising the following components:
Allothermal fluidized bed gasifier for the gasification of the biogenic input materials by means of impulse burners for the integrated generation of process heat;
Additional gasifier preferably connected in parallel, working according to the principle of the expanded or circulating fluidized bed, as a second gasifier for the gasification of inert pyrolytic coke from the allothermal fluidized bed gasifier using oxygen/steam as medium; and
Facilities for the combination of at least part of the gasification products from the two gasifiers for common processing.

9. The facility according to claim 8 incorporating means to screen and/or examine the discharged material to separate carbon and/or fine particles to isolate biological silicate contained in the ash.

10. The facility according to claim 8 incorporating means to return coarse particles of ash to the allothermal gasifier and/or discharge fine particles of ash as a high-quality biological silicate product.

11. The facility according to claim 8, in which the biological synthesis gas is to be processed by one or more of the following means:
In-situ desulfurization means;
Hot gas purification means;
Means for the removal of halogens by adsorption;
Means for 1- or 2-step fine cleaning using multi-cyclones and sintered metal filters;
Quenching means using a non-aqueous washing liquid to wash out traces of condensable aliphatic and/or aromatic hydrocarbons; and
Gas cooling means for subsequent compression steps.

12. The facility according to claim 8 incorporating means to produce dimethyl ether (DME) from generated biological synthesis gas via intermediate methanol.

13. The facility according to claim 8, in which inert pyrolytic coke produced in the allothermal gasifiers is reacted in a second gasification step by gasifiers working according to the principle of the expanded or circulating fluidized bed using oxygen/steam as gasification agents.

14. The facility according to claim 8 incorporating means by which, with regard to a molar CO/H₂ portion, previously compressed biological synthesis gas in a pure gas CO shift process is set such that an optimum ratio for further synthesis is achieved.

15. The use of a facility according to claim 8 for the production of fuel for a 2-stroke engine or a 4-stroke engine, in particular on a ship.

16. The use according to claim 15, characterized in that DME or synthesis gas is used in a 2-stroke version of an internal combustion box to generate electric power.

17. The use according to the claim 16, characterized in that the ash obtained during the generation of the synthesis gas is used to produce biological silica.

18. A process for the production of biological synthesis gases and/or a synthetic propellant, in particular DME (dimethyl ether), and/or biological silica using biogenic input materials and comprising the following steps:
allothermal gasification of the biogenic input material by means of impulse burners for the integrated generation of process heat in a fluidized bed gasifier;
gasification of inert pyrolytic coke from the allothermal gasification step in a second gasification step operated in parallel with the allothermal gasifier and further operated as an expanded or circulating fluidized bed using oxygen and/or steam as gasification agents; and
combination of at least part of the gasification products from the two gasifiers for common processing.
